(19) United States
(12) Patent Application Publication
(10) Pub. No.: US 2015/0080706 A1
(43) Pub. Date: Mar. 19, 2015

(54) Medical Imaging Device

(71) Applicants: Bernd Maciejewski, Markt Erlbach (DE); Sebastian Maier, Langenzenn/Lohe (DE); Alexander Müller, Altdorf (DE)

(72) Inventors: Bernd Maciejewski, Markt Erlbach (DE); Sebastian Maier, Langenzenn/Lohe (DE); Alexander Müller, Altdorf (DE)

(21) Appl. No.: 14/489,129

(22) Filed: Sep. 17, 2014

(30) Foreign Application Priority Data
Sep. 17, 2013 (DE) 102013218589.8

(51) Int. Cl. A61B 5/055; A61B 5/00

(52) U.S. Cl. CPC A61B 5/055; A61B 5/0035
USPC 600/411; 600/410

(57) ABSTRACT

A medical imaging device includes an imaging modality for recording a medical image, a housing unit for housing the imaging modality, a system electronics unit for operating the imaging modality, and a receiving element for accommodating the system electronics unit. The receiving element includes a connecting element for connecting the receiving element to the imaging modality and/or the housing unit.

This application claims the benefit of DE 10 2013 218 589.8, filed on Sep. 17, 2013, which is hereby incorporated by reference in its entirety.

### BACKGROUND

The present embodiments relate to a medical imaging device.

Medical imaging devices are widely used in clinical operation (e.g., in the field of diagnostics).

Medical imaging devices of this type may be bulky, and access to system electronics of the medical imaging devices may be associated with significant effort.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a medical imaging device that offers simplified access to the internal system electronics without negatively affecting the actual functionality is provided.

A medical imaging device includes an imaging modality for recording a medical image, a housing unit for housing the imaging modality, system electronics for operating the imaging modality, and a receiving element for accommodating the system electronics. The receiving element has a connecting element for connecting the receiving element to the imaging modality and/or the housing unit.

A medical imaging device is a device (e.g., an electronic and/or information-technology device) for acquiring, processing, evaluating and/or storing image information in the form of image data. In order to acquire image information, acoustic methods such as ultrasound (US), emission methods such as emission computed tomography (ECT) and positron emission tomography (PET), optical methods, and radiological methods such as x-ray tomography and computed tomography (CT) may be used, for example. The acquisition may, however, also take place using magnetic resonance tomography (MR or MRT) or using combined methods. The medical imaging device may deliver image data in a 2-dimensional (2D) or multi-dimensional such as 3-dimensional (3D) or 4-dimensional (4D) manner. The image data may be processed and/or stored in different formats. The medical imaging device may be used in diagnostics (e.g., in medical diagnostics).

An imaging modality includes the hardware elements used to record a medical image. A housing unit includes the casing elements that may be used to house the imaging modality. System electronics may be the electronic elements that are used to operate the imaging modality. A connecting element may include a mechanical element that facilitates access to the system electronics.

One or more of the present embodiments use a suitable arrangement of a receiving element to accommodate the system electronics. The receiving element has a connecting element for connecting the receiving element to the imaging modality and/or the housing unit.

Due to the construction of a medical imaging device of this type, the volume of the medical imaging device may be rendered useable to an optimum degree for the imaging modality and the housing unit, and also for the system electronics, by the system electronics being integrated into the receiving element and a user easily gaining access thereto by the connecting element. The design of this access also facilitates more user-friendly access to the system electronics from several sides. This also allows for improved lighting of the medical imaging device generally and, for example, of the system electronics, so that service activities on the system electronics may be implemented with improved visibility. The integration of the system electronics into the receiving element also results in an additional space saving depending on the embodiment of the medical device.

In one embodiment, the connecting element includes at least one slide bearing for moving the receiving element. In this way, the slide bearing may be embodied, for example, in the form of a rail or also in the form of one or a number of rolling elements. This allows for simplified access to the system electronics, by the receiving element being moveable by the connecting element. This further enables heavy hardware elements of the medical imaging device (e.g., a magnet of a magnetic resonance device) not to be subject to uneven loading, and existing damping elements may be configured equally. One or more of the present embodiments thus deliver high stability overall.

In one embodiment, the connecting element extends along a longitudinal direction of the medical imaging device. The longitudinal direction is also the direction in which the receiving element may be moved (e.g., an extension direction of the receiving element or a retraction direction of the receiving element). The longitudinal direction is also the direction in which a patient couch is inserted into the medical imaging device. Simplified access to the system electronics is enabled as a result.

In a further embodiment, the movement of the receiving element is effected along a longitudinal direction of the medical imaging device using the slide bearing. If the receiving element is moved by the slide bearing, even simpler and/or more rapid access to the system electronics may be achieved.

In one embodiment, the receiving element includes at last one wall unit for at least partially housing the system electronics. The housing unit may also enclose the wall unit. The additional housing provides protection of the system electronics, for example, from external influences such as dust or unauthorized access.

In one embodiment, at least one wall unit includes a design element. The design element may be, for example, a panel for decorative purposes, an illuminated panel, initials, lettering or another logo that is applied, attached, or inserted on at least one wall unit. The design element may be used, integrated and also replaced in a flexible manner by applying or attaching or insertion on at least one wall unit. For example, during a hardware revision of the medical imaging device, the design element may also be replaced, for example, by replacing the at least one wall unit. This is also cost-effective from a service point of view.

In one embodiment, the wall unit has a flat surface with at least one part of the housing unit or is arranged completely within the housing unit. An operating position of the receiving element may be a position that the receiving element adopts during operation of the medical imaging device. The formation of a flat surface may also include the formation of a homogenous surface. The formation of a flat surface provides a high degree of integration between the receiving element and the housing unit.

In a further embodiment, the wall unit, in a rest position of the receiving element, is arranged at least partially outside the housing unit. A rest position of the receiving element may be a position that the receiving element may adopt when the medical imaging device is not operating. The arrangement of the receiving element outside the housing unit facilitates simple access to the system electronics.

In one embodiment, the receiving element includes a handle element for gripping the receiving element. The handle element may be, for example, a handle, an arm, a lever, or a recess, by which the receiving element moves upon actuation of the handle element by an operator (e.g., may be extended or retracted). In such cases, the handle element may be attached to one edge of a front face of the receiving element in order to be able to embody the design element as large as a wall element, for example. Simpler and/or more rapid access to the system electronics may thus be achieved.

In one embodiment, the receiving element is arranged in a transverse direction adjacent to the imaging modality. Transverse direction may be the direction at right angles to the longitudinal direction, which does not run parallel, but, for example, runs at right angles to an acting force of gravity. Simple lateral system access may be provided as a result.

In a further embodiment, the medical imaging device includes a magnetic resonance device. This enables heavy hardware elements such as, for example, a magnet not to be subject to uneven loading, and existing damping elements may be configured equally. One or more of the present embodiments thus deliver high stability overall.

In one embodiment, the medical imaging device includes a combined magnetic resonance positron emission tomography device.

### BRIEF DESCRIPTION OF THE DRAWINGS

FIG. 1 shows an embodiment of a medical imaging device;

FIG. 2 shows a further embodiment of a medical imaging device from another viewing angle; and

FIG. 3 shows an embodiment of a medical imaging device from a further viewing angle.

### DETAILED DESCRIPTION

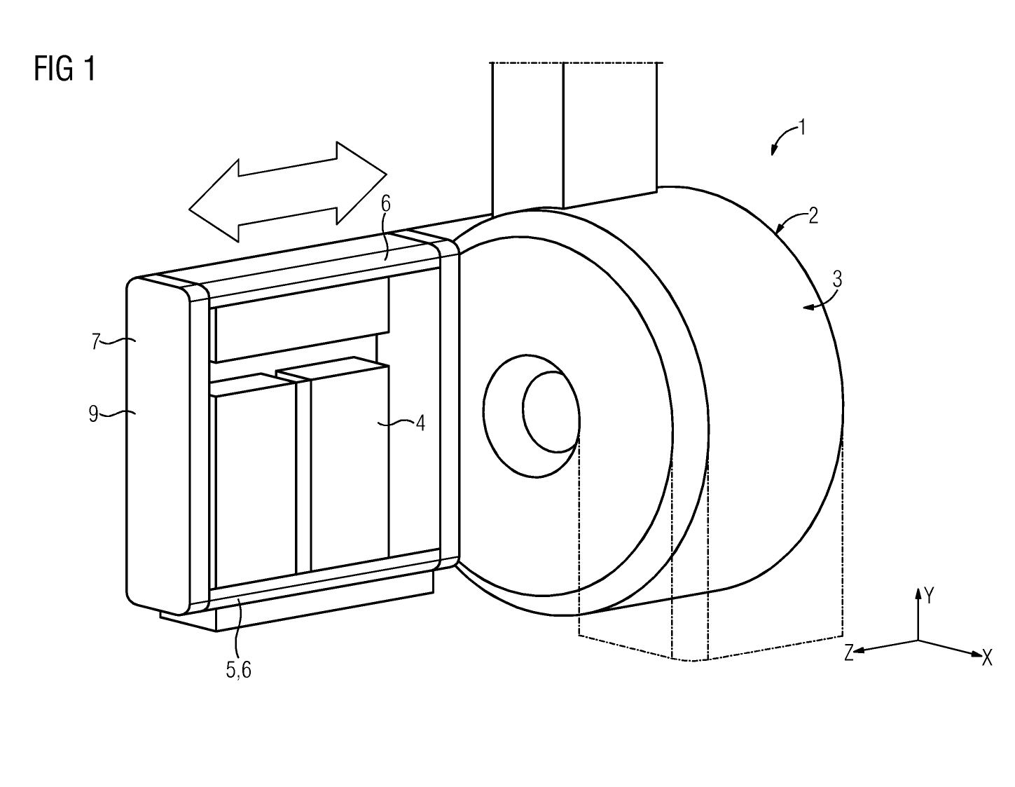

FIG. 1 shows an embodiment of a medical imaging device 1.

The medical imaging device 1 (e.g., configured as a magnetic resonance device) extends along an x-direction, along a y-direction, and along a z-direction. The z-direction is aligned parallel to a longitudinal direction, and the x-direction is aligned parallel to a transverse direction of the medical imaging device 1. The y-direction is aligned parallel to a force of gravity acting on the medical imaging device 1. The force of gravity acts in the negative y direction.

Alternatively, the medical imaging device 1 may also include a combined magnetic resonance positron emission tomography device, or other medical imaging devices 1.

The medical imaging device 1 includes an imaging modality 2 for recording a medical image, a housing unit 3 for housing the imaging modality 2, system electronics 4 for operating the imaging modality 2, and a receiving element 5 for accommodating the system electronics 4. The receiving element 5 includes a connecting element 6 for connecting the receiving element 5 to the imaging modality 2 and the housing unit 3.

The connecting element 6 (e.g., as a mechanical element) includes a slide bearing (e.g., in the form of a rail or also in the form of one or a number of rolling elements) for moving the receiving element 5 and extends along the longitudinal direction z of the medical imaging device 1. The longitudinal direction z is also the direction along which the receiving element 5 may be moved (e.g., an extension or retraction direction of the receiving element 5). The movement of the receiving element 5 along this longitudinal direction z takes place using the slide bearing. This facilitates simplified access to the system electronics 4 by the receiving element 5 being moveable in and counter to the positive z-direction using the connecting element 6.

In FIG. 1, the receiving element 5 includes a wall unit 7 for partially housing the system electronics 4. The receiving element 5 may also include more (e.g., six) wall units 7 in order to completely house the system electronics 4. In the example shown in FIG. 1, the wall unit 7 is surrounded by the housing unit 3. The wall unit 7 also includes a design element 9 (e.g., a panel for decorative purposes, an illuminated panel, initials, lettering or another logo). The design element 9 is applied, attached, or inserted on the wall unit 7. The design element 9 may be used, integrated and also replaced by applying, attaching, or by insertion on at least one wall unit 7.

In the view shown, the wall unit 7 is arranged at least partially outside the housing unit 3. This corresponds to a rest position of the receiving element 5 (e.g., a position that the receiving element 5 adopts when the medical imaging device 1 is not in operation). The wall unit 7 may, however, also have a flat surface with at least one part of the housing unit 3 or may also be arranged completely within the housing unit. This corresponds to an operating position of the receiving element 5 (e.g., a position that the receiving element 5 has during operation of the medical imaging device 1).

In the arrangement of the receiving element 5 in the rest position, facilitated access to the system electronics 4 from two sides is provided. Maintenance, service or control work may be performed under optimized conditions.

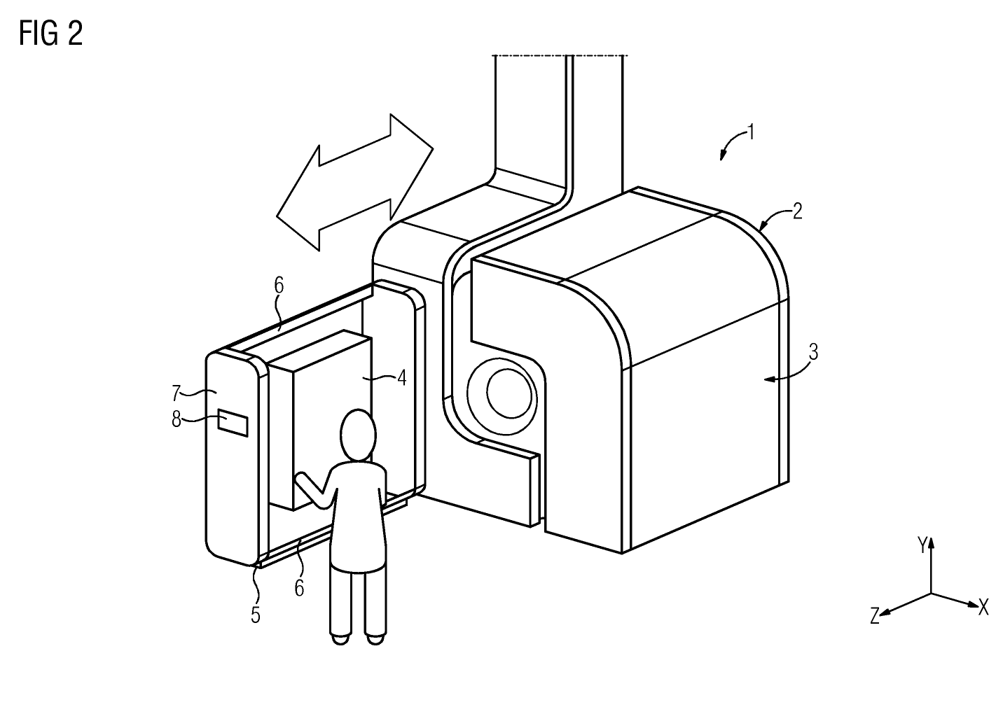

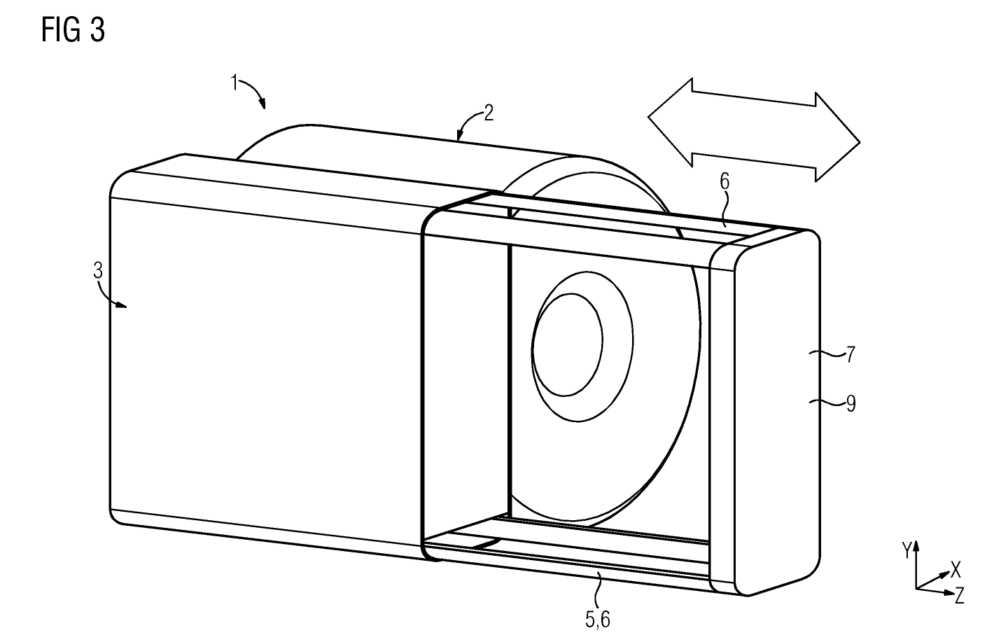

FIG. 2 and FIG. 3 indicate, relating to FIG. 1, exemplary embodiments of a medical imaging device 1 from other viewing angles. Essentially similar components, features and functions are basically numbered with the same reference characters. The subsequent descriptions are essentially restricted to the differences in the exemplary embodiment in FIG. 1, where with respect to similar components, features and functions, reference is made to the description of the exemplary embodiment in FIG. 1.

FIG. 2 shows a further embodiment of a medical imaging device 1 from another viewing angle. The corresponding spatial directions x, y, z along which the medical imaging device 1 extends may be inferred from the coordinate system.

In the embodiment shown, the receiving element 5 includes a handle element 8 for gripping the receiving element 5. The handle element 5 may be embodied as a handle, an arm, a lever or also a recess, for example, by which the receiving element may be removed and also reinserted. The facilitated access to the system electronics 4 from two sides is provided.

FIG. 3 shows a third embodiment of a medical imaging device 1 from a further viewing angle. The corresponding spatial directions x, y, z along which the medical imaging device 1 extends may be inferred from the coordinate system.

Although the invention is illustrated and described in more detail by the exemplary embodiments, the invention is not restricted by the disclosed examples, and other variations can be derived herefrom by the person skilled in the art without departing from the protective scope of the invention.

One or more of the present embodiments relate to a medical imaging device including an imaging modality for recording a medical image, a housing unit for housing the imaging modality, a system electronics unit for operating the imaging modality, and a receiving element for accommodating the system electronics. The receiving element includes a connecting element for connecting the receiving element with the imaging modality and/or the housing unit.

In an embodiment, the connecting element includes at least one slide bearing for moving the receiving element and extends along a longitudinal direction of the medical imaging device.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A medical imaging device comprising:
an imaging modality operable to record a medical image;
a housing unit for housing the imaging modality;
a system electronics unit operable to operate the imaging modality; and
a receiving element for accommodating the system electronics unit, wherein the receiving element includes a connecting element for connecting the receiving element to the imaging modality, the housing unit, or the imaging modality and the housing unit.

2. The medical imaging device of claim 1, wherein the connecting element includes at least one slide bearing operable to move the receiving element.

3. The medical imaging device of claim 1, wherein the connecting element extends along a longitudinal direction of the medical imaging device.

4. The medical imaging device of claim 2, wherein a movement of the receiving element takes place along a longitudinal direction of the medical imaging device using the slide bearing.

5. The medical imaging device of claim 1, wherein the receiving element includes at least one wall unit for at least partially housing the system electronics unit.

6. The medical imaging device of claim 5, wherein the at least one wall unit includes a design element.

7. The medical imaging device of claim 5, wherein the at least one wall unit, in an operating position of the receiving element, has a flat surface with at least one part of the housing unit or is arranged completely within the housing unit.

8. The medical imaging device of claims 5, wherein the at least one wall unit is arranged in a rest position of the receiving element at least partially outside the housing unit.

9. The medical imaging device of claim 1, wherein the receiving element includes a handle element for gripping the receiving element.

10. The medical imaging device of claim 1, wherein the receiving element is arranged in a transverse direction adjacent to the imaging modality.

11. The medical imaging device of claim 1, wherein the medical imaging device includes a magnetic resonance device.

12. The medical imaging device of claim 1, wherein the medical imaging device includes a combined magnetic resonance positron emission tomography device.

13. The medical imaging device of claim 2, wherein the connecting element extends along a longitudinal direction of the medical imaging device.

14. The medical imaging device of claim 13, wherein a movement of the receiving element takes place along the longitudinal direction of the medical imaging device using the slide bearing.

15. The medical imaging device of claim 14, wherein the receiving element includes at least one wall unit for at least partially housing the system electronics unit.

16. The medical imaging device of claim 15, wherein the at least one wall unit includes a design element.

17. The medical imaging device of claim 15, wherein the at least one wall unit, in an operating position of the receiving element, has a flat surface with at least one part of the housing unit or is arranged completely within the housing unit.

18. The medical imaging device of claims 15, wherein the at least one wall unit is arranged in a rest position of the receiving element at least partially outside the housing unit.

19. The medical imaging device of claim 15, wherein the receiving element includes a handle element for gripping the receiving element.

20. The medical imaging device of claim 15, wherein the receiving element is arranged in a transverse direction adjacent to the imaging modality.
